(19) United States
(12) Patent Application Publication
(10) Pub. No.: US 2024/0122892 A1
(43) Pub. Date: Apr. 18, 2024

(54) PHEROMONE DIFFUSING GEL

(71) Applicant: VIRBAC, Carros (FR)

(72) Inventors: Aurélie Girardin, Saint Laurent du Var (FR); Patricia Monginoux, Villeneuve Loubet (FR); Vitor Manuel Da Silva Pinto, Setúbal (PT); Vera Mónica Ramos Da Silva Raínho, Vialonga (PT); Cátia Filipa Mendes Rodrigues, Fajarda (PT); Cindy Novo de Carvalho, Gondomar (PT); Maria Helena Machado Parrula Silva, Montijo (PT)

(73) Assignee: VIRBAC, Carros (FR)

(21) Appl. No.: 18/263,826

(22) PCT Filed: Feb. 1, 2022
(86) PCT No.: PCT/EP2022/052271

§ 371 Date: Aug. 1, 2023

(30) Foreign Application Priority Data
Feb. 2, 2021 (FR) FR2100989

(51) Int. Cl. A61K 31/231; A61K 9/00; A61K 9/06; A61K 47/36; A61P 25/22

(52) U.S. Cl. CPC A61K 31/231; A61K 9/007; A61K 9/06; A61K 47/36; A61P 25/22

(57) ABSTRACT

The present invention relates to diffusers for dogs and cats, containing formulations capable of passively diffusing semiochemical compositions.

The present invention relates to diffusers for dogs and for cats containing formulations capable of passively diffusing semiochemical compositions.

The present invention relates in particular to devices containing formulations capable of diffusing semiochemical compositions effective on dogs or cats for a longer period of time than the devices known in the prior art and that do not need to be connected to a power supply in order to work. The present invention also relates to the diffusion formulations contained in such devices, in particular the diffuser gels comprising the semiochemical compositions effective on dogs and cats.

Devices capable of passively diffusing semiochemical compositions are known in the prior art. The semiochemical compositions are compounds or mixtures of compounds naturally secreted by animals to communicate within the same species. For example, during lactation, bitches secrete a pheromone to reassure their puppies when they are near them. Similarly, cats secrete, on their faces, facial pheromones that they deposit in known places to mark a familiar territory.

Numerous studies have been conducted to identify these compound mixtures and reproduce them synthetically.

For example, such compound mixtures are indicated to help dogs better manage stress-generating situations (separation, weaning, transport, fear of noise or fireworks, etc.) and allow them to better adapt to new situations.

The pet products in question, particularly for dogs and cats, are, for example, sprays, active diffusers (electric plug-ins heating a semiochemical composition dispersed or solubilized in an organic solvent), and collars. The active diffusers of the prior art (ADAPTIL® Calm for dogs) comprise a reservoir containing a semiochemical composition dispersed or solubilized in an organic solvent in which a wick is immersed. When the device is plugged in, the wick is heated, which causes the semiochemical composition to diffuse into the animal environment through this wick; the semiochemical composition consists of a mixture of fatty acid methyl esters or a mixture of fatty acid methyl esters and fatty acids. This active diffuser is effective for four weeks.

One major drawback of this type of device used for dogs or cats is that it requires a power supply to activate it and activate the diffusion of the semiochemical composition. Moreover, it cannot be used in certain places where dogs and cats are present.

Devices for passive diffusion of semiochemical compositions have been used for farm animals. In pigs, for example, there are blocks comprising PAP or “Pig Appeasing Pheromones”, which are used on stock farms by positioning them in a stock pen at a height of 1.5 meters throughout their use in order to reduce aggression and ensure the welfare of weanling groups (Temple et al., Porcine Health Management 2, 13 (2016)). However, the composition of the slow-release blocks contained in these livestock devices is not described and their efficacy is limited to six weeks.

The Inventors have developed diffusion formulations contained in portable devices that can be moved freely without restrictions, in the form of a diffuser gel comprising a semiochemical composition comprising a mixture of volatile fatty acid derivatives; this diffuser gel allows for efficient passive diffusion of the semiochemical composition into the environment; this formulation thus makes it possible to dispense with the need for a power supply when using a diffuser incorporating the formulation and it can thus be carried and used in any location. In addition, it has a longer efficacy period compared to the diffusers of the prior art.

Moreover, the semiochemical composition formulated in this diffusion formulation displays good stability despite its components' fragility when exposed to humidity and light, and the passive diffusion device consists of a container that advantageously makes it possible to control the diffusion of the semiochemical composition.

The present invention therefore aims to improve on the devices of the prior art, in particular diffusers for dogs and/or for cats, comprising a semiochemical composition comprising a mixture of volatile fatty acid derivatives.

Unless otherwise specified, all percentages are listed by mass of a component relative to the total mass of the listed components (m/m).

A first purpose of the present invention is to provide a passive diffuser gel comprising:
a polysaccharide gel matrix,
an appeasing semiochemical composition for dogs and/or for cats, comprising a mixture of volatile fatty acid derivatives,
a solubilizer of said semiochemical composition in said gel matrix,
an organic acid having a pKa greater than 3,
optionally, a preservative,
optionally, a colorant,
optionally, a bittering agent.

The polysaccharide gel matrix is an aqueous polysaccharide gel; it is thus composed of a polysaccharide gelling agent and water; the polysaccharide gelling agent can be K-carrageenan, 1-carrageenan, A-carrageenan, agarose (agar), gellan gum, amylose gum, curdlan gum, alginate, xanthan gum, chitosan and deacetylated rhamsan gum or a mixture of these polysaccharide gelling agents.

According to a particular embodiment, the gel matrix is prepared from carrageenan; preferably, according to this embodiment, the carrageenan is present in a content comprised between 0.5 and 5%, preferentially between 1 and 2%, by weight relative to the total weight of the diffuser gel.

In order to obtain a gel having an appropriate viscosity that has the advantages of the invention in terms of stabilizing the semiochemical composition and of controlled and continuous release of adequate amounts of the semiochemical compounds, it is necessary to adjust the pH of the gel, in particular using an organic acid having a pKa of greater than 3. The organic acid can be any organic acid but citric acid will preferably be used, which proves to be particularly suitable for the semiochemical compositions according to the invention. The organic acid having a pKa greater than 3, in particular citric acid, is generally present at a content comprised between 0.01 and 0.5%, preferably about 0.05%, by mass of the total mass of the diffuser gel.

The semiochemical composition used is an appeasing semiochemical composition for dogs and/or for cats comprising a mixture of volatile fatty acid derivatives.

Within the meaning of the present invention, “appeasing semiochemical composition” refers to a mixture of naturally-occurring chemical substances having a signal value between living beings, for example extracted from a living being, or synthetically-occurring, for example obtained by mixing volatile derivatives of a fatty acid capable of producing a relaxing or appeasing effect on the animal.

Within the meaning of the present invention, “volatile derivative of a fatty acid” refers to any derivative of a fatty acid that can be diffused into the environment of the animal without applying an external heat source to the device.

Within the meaning of the present invention, “fatty acid” refers to a straight or branched, saturated or unsaturated, hydrocarbon-chain monocarboxylic or dicarboxylic acid having between 4 and 22 carbon atoms. Advantageously, the fatty acid is selected from the group consisting of oleic acid, palmitic acid, azelaic acid, pimelic acid, capric acid, lauric acid, myristic acid, palmitoleic acid, linoleic acid, stearic acid, arachidonic acid, n-butyric acid, isobutyric acid, α-methylbutyric acid, caproic acid, pivalic acid, γ-linoleic acid, eicosapentaenoic acid, pentadecanoic acid, tridecanoic acid, or docosahexaenoic acid.

The volatile fatty acid derivatives are in particular fatty acid esters, such as esters formed from fatty acid and from a C₁ to C₆ alcohol. Advantageously, the fatty acid ester is ethyl or methyl ester. Preferably, the fatty acid ester is fatty acid methyl ester.

Advantageously, the appeasing semiochemical composition for dogs and/or for cats therefore comprises a mixture of volatile fatty acid derivatives wherein the fatty acid is selected from the group consisting of oleic acid, palmitic acid, azelaic acid, pimelic acid, capric acid, lauric acid, myristic acid, palmitoleic acid, linoleic acid, stearic acid, arachidonic acid, n-butyric acid, isobutyric acid, α-methylbutyric acid, caproic acid, pivalic acid, γ-linoleic acid, eicosapentaenoic acid, pentadecanoic acid, tridecanoic acid, or docosahexaenoic acid, advantageously selected from the group consisting of oleic acid, palmitic acid, lauric acid, myristic acid, linoleic acid, stearic acid, and pentadecanoic acid.

More advantageously, the appeasing semiochemical composition for dogs and/or for cats comprises or consists of a mixture of volatile fatty acid derivatives selected from the group consisting of methyl oleate, methyl palmitate, methyl azelate, methyl pimelate, methyl caprate, methyl laurate, methyl myristate, methyl palmitoleate, methyl linoleate, methyl stearate, methyl arachidonate, methyl n-butyrate, methyl isobutyrate, methyl α-methylbutyrate, methyl caproate, methyl pivalate, methyl γ-linoleate, methyl eicosapentaenoate, methyl pentadecanoate, methyl tridecanoate, methyl docosahexaenoate, advantageously selected from the group consisting of methyl oleate, methyl palmitate, methyl laurate, methyl myristate, methyl linoleate, methyl stearate and methyl pentadecanoate.

The volatile derivatives of fatty acids and their relative proportions in the appeasing semiochemical composition depend on the target animal.

When the target animal is a dog, the appeasing semiochemical composition for dogs and/or for cats can be selected from those described in international application WO2009144321.

Particular mixtures of fatty acid esters, preferably of methyl esters, intended to appease dogs are esters of the following fatty acids:
a mixture A of oleic acid and of n-butyric acid;
a mixture B of oleic acid, of palmitic acid and of linoleic acid;
a mixture C of oleic acid, of palmitic acid, of linoleic acid and of palmitoleic acid;
a mixture D of capric acid, of lauric acid, of myristic acid, of palmitoleic acid, of palmitic acid, of linoleic acid and of oleic acid;
a mixture E of oleic acid, of palmitic acid, of linoleic acid and of myristic acid;
a mixture F of oleic acid, of palmitic acid, of linoleic acid, of lauric acid and of myristic acid;
a mixture G of oleic acid, of palmitic acid, of linoleic acid, of myristic acid and of pentadecanoic acid;
a mixture H of oleic acid, of palmitic acid, of linoleic acid, of myristic acid, of pentadecanoic acid and of stearic acid;
a mixture I of oleic acid, of palmitic acid, of linoleic acid, of myristic acid, of lauric acid and of pentadecanoic acid;
a mixture J of lauric acid, of myristic acid, of pentadecanoic acid, of palmitic acid, of stearic acid, of oleic acid, and of linoleic acid;
a mixture K of oleic acid, of azelaic acid, of pimelic acid and of palmitic acid.

Mixtures of fatty acid esters, preferably methyl esters, intended to appease dogs are esters of the following fatty acids:
mixture L comprising or consisting of about 55-65% of oleic acid, and 45-35% of palmitic acid;
mixture M comprising or consisting of about 45% of oleic acid, 16% of azelaic acid, 18% of pimelic acid, and 21% of palmitic acid;
mixture N comprising or consisting of about 30% of palmitic acid, 30% of oleic acid, and 40% of linoleic acid;
mixture O comprising or consisting of about 30% of palmitic acid, 40% of linoleic acid, 10% of palmitoleic acid, and 20% of oleic acid.

A particular mixture P of fatty acid esters for appeasing dogs comprises or consists of about 35% of methyl oleate, about 2% of methyl laurate, about 13% of methyl stearate, about 21% of methyl linoleate, about 5% of methyl myristate, about 4% of methyl pentadecanoate, and about 20% of methyl palmitate.

The Inventors of the present invention have also demonstrated that a semiochemical composition comprising or consisting of a mixture of volatile fatty acid derivatives comprising no methyl pentadecanoate led to a similar effect in the dog to that of the P mixture. As pentadecanoic acid is a relatively rare fatty acid in its natural state, as its primary source is the fatty fraction of cow's milk where it is present in a small amount (1.2%), it is difficult to obtain at a reasonable cost. The discovery that the absence of this fatty acid does not diminish the appeasing efficacy of a semiochemical composition therefore makes it possible to reduce the costs associated with the manufacture of the devices according to the invention. Another particular mixture O of fatty acid esters for appeasing dogs according to the invention comprises or consists of between 32% and 38% of methyl oleate, between 1 and 3% of methyl laurate, between 13 and 16% of methyl stearate, between 18 and 24% of methyl linoleate, between 3 and 7% of methyl myristate and between 18 and 24% of methyl palmitate. A more particular mixture R of fatty acid esters for appeasing dogs according to the invention comprises or consists of about 35% of methyl oleate, about 2% of methyl laurate, about 15.5% of methyl stearate, about 21% of methyl linoleate, about 5% of methyl myristate and about 21% of methyl palmitate.

When the target animal is a cat, the appeasing semiochemical composition can be selected from those described in international application WO2015140631.

The appeasing semiochemical composition is, for example, an appeasing pheromone for cats which is derived from skin secretions from the area between the shoulder blades and the ears of cats.

The semiochemical composition for appeasing cats advantageously comprises or consists of methyl palmitate, of methyl linoleate, of methyl oleate, of methyl stearate, of methyl laurate and of methyl myristate.

A particular mixture S of fatty acid esters for appeasing cats comprises or consists of between 15% and 25% of methyl palmitate, between 10% and 27% of methyl linoleate, between 25% and 35% of methyl oleate, between 5% and 15% of methyl stearate, between 5% and 15% of methyl laurate and between 5% and 15% of methyl myristate. Another particular mixture T of fatty acid esters for appeasing cats comprises or consists of between 19% and 26% of methyl palmitate, between 14% and 20% of methyl linoleate, between 26% and 32% of methyl oleate, between 8% and 14% of methyl stearate, between 8% and 14% of methyl laurate and between 8% and 14% of methyl myristate.

An even more particular mixture U comprises or consists of 22% methyl palmitate, 17% of methyl linoleate, 28% of methyl oleate, 11% of methyl stearate, 11% of methyl laurate and 11% of methyl myristate.

Another semiochemical composition V that can be used in the devices according to the invention is described in patent U.S. Ser. No. 10/227,321, wherein at least one volatile fatty acid derivative is an ester of the following formula:

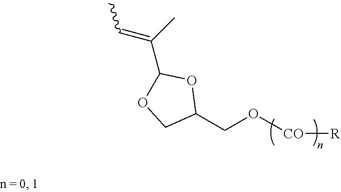

wherein n=0 and R is a saturated or unsaturated straight alkyl comprising from 9 to 30 carbon atoms or n=1 and R is a saturated or unsaturated straight alkyl comprising from 8 to 22 carbon atoms.

The amount of semiochemical composition in the device may vary in large proportions. The proportion of the semiochemical composition is typically comprised between 0.1% and 25% by mass of the total mass of the diffuser gel. Advantageously, the semiochemical composition represents from 1 to 10% by mass, more advantageously from 3 to 8% by mass, preferably about 6% of the total mass of the diffuser gel.

Within the meaning of the present invention, “about x %” refers to a variation of about 0.2% from the stated percentage.

As the semiochemical composition consists of water-immiscible molecules and due to the high water content of the gel, the diffuser gel according to the invention comprises at least one solubilizer of the semiochemical composition in the gel matrix, in order to avoid the formation of two phases, first one consisting of water and the second one of the semiochemical composition (demixing).

The role of the solubilizer is also to facilitate evaporation of the components of the semiochemical composition. This evaporation influences the release of the semiochemical composition and therefore the efficacy of the device according to the invention.

The solubilizer may be at least one surfactant, at least one water-miscible organic solvent or a mixture of at least one surfactant and at least one water-miscible organic solvent.

The surfactant can be selected from anionic, non-ionic, cationic or amphoteric surfactants.

Anionic surfactants include acyl amino acids (and salts); carboxylic acids (and salts), phosphoric acids (and salts); sulfonic acids (and salts), alkyl aryl sulfonates, alkyl sulfonates, alkyl sulfosuccinates (and salts), and sulfuric acid esters, such as alkyl ether sulfates, alkyl sulfates, alkyl phenol ethoxylates, and alkyl phenol sulfates.

Nonionic surfactants include aliphatic alcohol ethoxylates; polyoxyethylenes; carboxylic esters; polyethylene glycol esters; anhydrosorbitol esters and ethoxylated derivatives thereof; fatty acid glycol esters; carboxylic amides; polyoxyethylene fatty acid amides.

Cationic surfactants mainly include long chain quaternary alkyl ammonium salts.

Amphoteric surfactants include zwitterionic surfactants such as betaines (for example, alkyl betaines, alkyl amido betaines, sulfobetaines, sulfitobetaines, sulfatobetaines, phosphinate betaines, phosphonate betaines, phosphitobetaines, phosphatobetaines, sulfonium betaines and phosphenium betaines).

The water-miscible organic solvent can be any water-miscible solvent in which the semiochemical composition is soluble.

Among the organic solvents miscible with water, reference can be made to alcohols such as methanol, ethanol, 1-propanol, 2-propanol, 1-butanol, 2-butanol and tert-butanol; ketones such as acetone; ethers such as dioxane or dimethoxyethane; sulfoxides such as dimethyl sulfoxide; amides such as dimethylformamide or N-Methyl-2-pyrrolidone; nitriles such as acetonitrile; polyols such as 1,2-butanediol, 1,3-butanediol, 1,3-propanediol, 1,4-butanediol, 1,5-butanediol, ethylene glycol, propylene glycol, glycerol or triethylene glycol.

Advantageously, the solubilizer is a water-miscible organic solvent, in particular selected from C1 to C4 alcohols and polyols. Preferably, the C1 to C4 alcohol is 1-propanol or 2-propanol, more preferentially 2-propanol.

The smallest possible amount of solubilizer is used. Generally, a solubilizer content comprised between 1 and 10% by mass of the total mass of the diffuser gel makes it possible to obtain a gel that is homogeneous and stable over time, in which no demixing is observed.

When the solubilizer is a C1 to C4 alcohol or a polyol, in particular 1-propanol or 2-propanol, more preferentially 2-propanol, it is present at a content comprised between 1 and 10%, advantageously between 2 and 8%, preferably about 4%, by mass of the total mass of the diffuser gel.

Any preservative, in particular biocide-type preservatives, can be added to the diffuser gel; this family of products is well known to the person skilled in the art; it comprises in particular isothiazolinones (benzoisothiazolinone, isothiazolinone, methylchloroisothiazolinone, n-octyl-isothiazolinone, etc.), quaternary ammoniums (benzalkonium).

According to a particular embodiment, the preservative is benzoisothiazolinone and is present at a content comprised between 0.05 and 1%, preferably about 0.18%, by mass of the total mass of the diffuser gel.

In addition, the stability of the diffuser gel according to the present invention and in particular the stability of the components of the semiochemical composition, can be further improved by incorporating antioxidants. Among the antioxidants, reference can be made to phenolic compounds such as butyl hydroxyanisole (BHA), butyl hydroxytoluene (BHT), gallic acid, propyl gallate and pyrogallol, hydroquinones such as tert-butylhydroguinone (TBHQ) and di-tert-butylhydroquinone (DTBHQ), tocopherols, amines such as octyl butyl diphenylamine (OBPA). poly(1,2-dihydro-2,2,4-trimethylguinoline (Orox PK), N,N′-phenyl-p-phenylenediamine (DPD) and ethoxyguin, and a mixture of these antioxidants. Such compounds are well known to the person skilled in the art and can be selected without undue effort; preferably, the antioxidant used is tBHQ, For information purposes, the amount of antioxidants used may be comprised between 0.001 and 0.1%,

In order to make the visual appearance of the diffuser gel more appealing, any colorant can be added to it.

Lastly, to avoid accidental ingestion of the diffuser gel, by a child or an animal, for example, a bittering agent can be added to it; these agents are known to the person skilled in the art, such as denatonium benzoate; this compound can be added in a content comprised between 0.05 and 0.15% by mass relative to the total mass of the diffuser gel.

The present invention further relates to a diffuser gel comprising:
a polysaccharide gel matrix;
from 1 to 10% of an appeasing semiochemical composition for dogs and/or for cats comprising a mixture of volatile fatty acid derivatives as defined above, preferably about 6%; in particular, the semiochemical composition can comprise a mixture of fatty acid esters selected among laurate, myristate, palmitate, stearate, oleate and linoleate, and optionally pentadecanoate;
from 1 to 10% of a solubilizer of said semiochemical composition in said gel matrix, preferably about 4%;
between 0.1 and 1% of an organic acid having a pKa greater than 3, in particular citric acid;
optionally, from 0.1 to 0.5% of a preservative;
optionally, a colorant;
optionally, a bittering agent.

In a specific embodiment, the device according to the present invention comprises:
a gel matrix consisting of an aqueous carrageenan gel;
from 1 to 10% of an appeasing semiochemical composition as defined above, preferably about 6%; in particular, the semiochemical composition can comprise a mixture of fatty acid esters selected among laurate, myristate, palmitate, stearate, oleate and linoleate, and optionally pentadecanoate;
from 1 to 10% of a solubilizer of said semiochemical composition in said gel matrix, selected from C1 to C4 alcohols, preferably about 4%;
between 0.1 and 1% of citric acid;
optionally, from 0.1 to 0.5% of a preservative from the isothiazolinone family;
optionally, a colorant;
optionally, denatonium benzoate as a bittering agent.

In a specific embodiment, the device according to the present invention comprises:
a gel matrix comprising an aqueous carrageenan gel;
from 1 to 10% of a semiochemical composition, said semiochemical composition being a mixture of fatty acid esters comprising between 32% and 38% of methyl oleate, between 1 and 3% of methyl laurate, between 13 and 16% of methyl stearate, between 18 and 24% of methyl linoleate, between 3 and 7% of methyl myristate and between 18 and 24% of methyl palmitate, preferably about 6%;
from 2 to 10% of a solubilizer of said semiochemical composition in said gel matrix selected from C1 to C4 alcohols;
between 0.1 and 1% of citric acid;
from 0.1 to 0.5% of a preservative from the isothiazolinone family;
a colorant;
denatonium benzoate as a bittering agent.

A second purpose of the present invention is to provide a diffuser containing a diffuser gel as described according to the first purpose of the invention, allowing the semiochemical composition to diffuse into the environment of the dog and/or of the cat, which will then produce its appeasing effect on the animal.

The diffuser is a container capable of holding a diffuser gel comprising:
a polysaccharide gel matrix,
an appeasing semiochemical composition for dogs and/or for cats comprising a mixture of volatile fatty acid derivatives,
a solubilizer of said semiochemical composition in said gel matrix,
an organic acid having a pKa greater than 3,
optionally, a preservative,
optionally, a colorant,
optionally, a bittering agent.

The diffuser is a container for holding the diffuser gel and for allowing controlled release of the semiochemical composition. It is capable of having any shape and necessarily comprises at least one diffuser gel-to-air exchange area, so that the appeasing semiochemical composition for dogs and/or for cats diffuses into the air around the diffuser. Thus, positioning the diffuser in the vicinity of the dog and/or of the cat allows the cat and/or the dog to be in contact with the appeasing semiochemical composition. The diffusers can be carried or transported or even traveling, meaning that they can be transported and positioned by the dog or cat owner from place to place wherever the pets are present in order to produce their effect. The diffusers according to the invention will be primarily set in enclosed places in which the dog or cat is present in order to avoid unnecessary wind dispersion of the semiochemical compounds. These include, for example, living rooms, vehicles (cars) or the waiting room at a veterinary clinic.

The diffuser gel-to-air exchange area is preferably located on the top of the container in order to avoid any risk of the diffuser gel leaking out of the container.

The diffuser gel-to-air exchange area is advantageously made of a wall of the container in which one or a plurality of openings through which the semiochemical composition can diffuse are arranged. The openings can have simple geometric shapes such as circles, squares, diamonds, rectangles and triangles or have shapes with an aesthetic function such as the head of the animal for which the device is intended, the silhouette of the body of the animal for which the device is intended or even the paw of the animal for which the device is intended. This type of visual indication, when it identifies the target animal, the dog or the cat, can be a valuable aid for an elderly owner in the proper use of the diffuser.

Advantageously, the surface in contact with the air at the diffuser gel-to-air exchange area is controlled by adjustable means of closing the openings. These adjustable closure means make it possible, by controlling the diffuser gel-to-air exchange area, to reduce or increase the rate of diffusion of the semiochemical composition in the environment according to the animal's need and/or to the size of the room. The required diameter of the openings and the number of openings needed to achieve adequate diffusion can be determined experimentally.

The diffuser gel-to-air exchange area is advantageously covered by a removable, airtight lid that makes it possible to stop the diffusion of the semiochemical composition into the air when the lid in place. Thus, the natural diffusion or evaporation of the semiochemical composition can be greatly reduced or even stopped, which is particularly useful as it is economic and ecological, when the diffuser is no longer in the vicinity of the dog or of the cat (when the dog or cat is on a walk, for example, or when administering the appeasing composition is not required).

In a particular embodiment incorporating the characteristics described above, the invention relates to a container having a bottom wall, a side wall, a top wall or a diffuser gel-to-air exchange area having a plurality of openings and a lid having a plurality of openings capable of rotating relative to the top wall in order to align or misalign the plurality of openings in the top wall and the plurality of openings in the lid. In this embodiment, the top wall and lid define a system for controlling the diffusion of the semiochemical composition by increasing, decreasing, or ending the diffuser gel-to-air exchange in the animal's environment. Such a system for controlling the surface of the diffuser gel-to-air exchange makes it possible to adapt the release to the size of the room in which the animal is present and to increase the useful life of the diffuser by ensuring the stability of the semiochemical composition containing the diffuser gel once the diffuser is tightly closed when its use is unnecessary.

According to another particular embodiment, the container has a double lid, the first lid having holes and being attached directly to the container, the second lid attached above the first and enabling the openings of the first lid to be closed; to use the device in this embodiment, when the user has chosen the appropriate place to set the device, he or she can open the second lid and the semiochemical composition can then passively diffuse the diffuser gel into the air through the openings in the first lid.

To facilitate the transport and/or storage of diffuser gel packaged in a container, the container can comprise an airtight closure means, such as a lid and/or a seal. For ease of transport, a handle can be provided on the diffuser. Optionally, the device can also comprise a means of attachment such as a hook, an adhesive strip, etc.

The present invention also relates to a method for preparing a diffuser gel as previously defined, comprising the steps of:
1) heating, to a sufficient temperature and for a sufficient time to solubilize the polysaccharide gelling agent, a mixture comprising:
   a polysaccharide gelling agent,
   a semiochemical composition,
   a solubilizer of said semiochemical composition,
   an organic acid having a pKa greater than 3,
   optionally, a preservative,
   optionally, a colorant,
   optionally, a bittering agent,
1) cooling the solution obtained in step 1) so as to obtain a diffuser gel as previously defined.

The diffuser gel according to the invention is advantageously prepared by mixing the semiochemical composition and the solubilizer of the semiochemical composition so as to obtain a first pre-mix.

All the mixing and/or heating steps are preferably carried out under stirring.

A second pre-mix comprising water, the organic acid having a pKa greater than 3, the colorant and the bittering agent is prepared. Advantageously, the second pre-mix is prepared by first mixing the water and the organic acid having a pKa greater than 3, optionally the colorant and the bittering agent, and heating to a sufficient temperature and for a sufficient time so as to obtain a first solution in which the ingredients are completely solubilized. Typically, this first solution is obtained by heating to a temperature comprised between 50 and 100° C., in particular to a temperature of about 70° C.

To this first solution is then added the polysaccharide gelling agent and the mixture is heated to a sufficient temperature and for a sufficient time so as to obtain a second solution in which the polysaccharide gelling agent is completely dissolved. As polysaccharide gelling agents can degrade when heated to too high a temperature and/or for too long a time, it is therefore necessary to heat the mixture to an appropriate temperature and for an appropriate amount of time.

The temperature and the heating time can be determined by routine tests. Typically, when the polysaccharide gelling agent is a carrageenan, the solution is heated to a temperature of about 70° C. for at least 1 hour. A simple visual check makes it possible to verify that the polysaccharide gelling agent is completely dissolved.

When the polysaccharide gelling agent is fully dissolved, the first pre-mix is added to the first solution.

The first pre-mix can be added at the same temperature as the one at which the first solution is prepared or at a different temperature. In an advantageous embodiment, the first pre-mix is added at a temperature lower than the temperature at which the first solution is prepared but at which the polysaccharide gelling agent is always solubilized. When a preservative is added, it is added to the first solution, advantageously after the first pre-mix.

After adding the first pre-mix and optionally the preservative to the first solution, a second solution is obtained comprising:
a polysaccharide gelling agent,
a semiochemical composition,
a solubilizer of said semiochemical composition,
an organic acid having a pKa greater than 3,
optionally, a preservative,
optionally, a colorant,
optionally, a bittering agent.

Said second solution is kept at a temperature sufficient to allow the formation of a homogenous solution that can be filled into diffusers, as described above. By cooling to a temperature of about 15° C. to 25° C. (room temperature), the second solution solidifies to form a diffuser gel as described above. Alternatively, the homogenous solution can be directly cooled and the gel block formed after cooling at room temperature can be divided into as many parts as desired.

In an advantageous embodiment, the present invention relates to a method for preparing a diffuser gel as previously defined, comprising the steps of:
1) mixing a semiochemical composition and a solubilizer of the semiochemical composition so as to obtain a first pre-mix,
2) mixing water, an organic acid having a pKa greater than 3, optionally a colorant and a bittering agent, and heating to a sufficient temperature and for a sufficient time so as to obtain a first solution,
3) adding a polysaccharide gelling agent to the first solution and heating, to a sufficient temperature and for a sufficient time, so as to obtain a second pre-mix in which the polysaccharide gelling agent is completely dissolved,
4) adding the first pre-mix obtained in step 1), and optionally a preservative, to the first solution obtained in step 3), and heating the mixture so as to obtain a homogeneous solution,
5) cooling the homogenous solution obtained in step 4 so as to obtain a diffuser gel as previously defined.

In a preferred embodiment, the present invention relates to a method for preparing a diffuser gel as previously defined, comprising the steps of:
1) mixing a semiochemical composition and isopropanol, so as to obtain a first pre-mix,
2) mixing water, citric acid, a colorant and a bittering agent, and heating to a sufficient temperature and for a sufficient time so as to obtain a first solution,
3) adding a carrageenan to the first solution and heating it to a sufficient temperature and for a sufficient time, so as to obtain a second solution in which the carrageenan is completely dissolved,
4) adding the first pre-mix obtained in step 1), and optionally the preservative, to the second solution obtained in step 3), and heating the mixture so as to obtain a homogeneous solution,
5) cooling the homogenous solution obtained in step 4) so as to obtain a diffuser gel as previously defined.

The present invention also relates to a method for obtaining one or more of the following effects:
ensure the welfare of an animal, in its environment,
help an animal handle stressful and/or anxiety-generating situations,
reduce separation-related stress in an animal,
reduce weaning-related stress in an animal,
reduce solitude-related stress in an animal,
reduce stress in an animal before and/or during transport,
reduce stress associated with noise such as thunderstorms or fireworks,
reduce stress associated with a medical condition, a medical treatment, a veterinary visit that may require auscultation or even surgery,
reduce aggressiveness in an animal,
improve an animal's behavior, particularly an aggressive animal or a fearful animal,
produce a relaxing effect on the animal,
help an animal adapt to a new situation, for example upon adoption, when a new animal or baby arrives in its environment, when moving house,
reduce stress-related urinary marking in an animal,
reduce an animal's housebreaking issues,
improve the conditions in which an animal becomes familiar with other animals of the same species,
prevent scratching or destruction of the territory,
reduce noisy manifestations such as whining, barking,
achieve less aggressive, more relaxed and more affectionate behavior in an animal, especially with the owners,

comprising the diffusion of a semiochemical composition from a diffuser as described above, advantageously for a period of more than one month, more advantageously more than two months, preferably for a period of about three months, the animal being a dog or a cat.

In another aspect, the present invention relates to the use of a diffuser gel according to the invention or of a diffuser as described above to treat stress, anxiety and/or aggressive behavior in a cat or in a dog and/or to obtain one or more of the following effects:
ensure the welfare of an animal, in its environment,
help an animal handle stressful and/or anxiety-generating situations,
reduce separation-related stress in an animal,
reduce weaning-related stress in an animal,
reduce solitude-related stress in an animal,
reduce stress in an animal before and/or during transport,
reduce stress associated with noise such as thunderstorms or fireworks,
reduce stress associated with a medical condition, a medical treatment, a veterinary visit that may require auscultation or even surgery,
reduce aggressiveness in an animal,
improve an animal's behavior, particularly an aggressive animal or a fearful animal,
produce a relaxing effect on the animal,
help an animal adapt to a new situation, for example upon adoption, when a new animal or baby arrives in its environment, when moving house,
reduce stress-related urinary marking in an animal,
reduce an animal's housebreaking issues,
improve the conditions in which an animal becomes familiar with other animals of the same species,
prevent scratching or destruction of the territory,
reduce noisy manifestations such as whining, barking,
achieve less aggressive, more relaxed and more affectionate behavior in an animal, especially with the owners, advantageously for a period of more than one month, more advantageously more than two months, preferably for a period of about three months, the animal being a dog or a cat.

Lastly, the present invention relates to a device according to the invention, for its use in the treatment of stress, anxiety and/or aggressive behavior in a cat or in a dog.

The present invention further relates to a non-therapeutic method of appeasing an animal, particularly to:
ensure the welfare of an animal, in its environment,
help an animal handle stressful and/or anxiety-generating situations,
reduce separation-related stress in an animal,
reduce weaning-related stress in an animal,
reduce solitude-related stress in an animal,
reduce stress in an animal before and/or during transport,
reduce stress associated with noise such as thunderstorms or fireworks,
reduce stress associated with a medical condition, a medical treatment, a veterinary visit that may require auscultation or even surgery,
reduce aggressiveness in an animal,
improve an animal's behavior, particularly an aggressive animal or a fearful animal,
produce a relaxing effect on the animal,
help an animal adapt to a new situation, for example upon adoption, when a new animal or baby arrives in its environment, when moving house,
reduce stress-related urinary marking in an animal,
reduce an animal's housebreaking issues,
improve the conditions in which an animal becomes familiar with other animals of the same species,
prevent scratching or destruction of the territory,
reduce noisy manifestations such as whining, barking,
achieve less aggressive, more relaxed and more affectionate behavior in an animal, especially with the owners,

comprising the use of a diffuser according to the invention in the environment of said animal.

The present invention also relates to a method for treating stress, anxiety and/or aggressive behavior in a cat or in a dog, said method consisting of opening the diffuser gel-to-air exchange area of a diffuser according to the invention.

The present invention further relates to a semiochemical composition as defined above for preventing manifestations of stress and/or reducing anxiety in a pet for a period of more than one month, more advantageously more than two months, preferably for a period of about three months, characterized in that the semiochemical composition is present in a diffuser gel according to the invention.

In particular, it relates to a semiochemical composition as defined above for its use in appeasing an animal, particularly to:
ensure the welfare of an animal, in its environment,
help an animal handle stressful and/or anxiety-generating situations,
reduce separation-related stress in an animal,
reduce weaning-related stress in an animal,
reduce solitude-related stress in an animal,
reduce stress in an animal before and/or during transport,
reduce stress associated with noise such as thunderstorms or fireworks,
reduce stress associated with a medical condition, a medical treatment, a veterinary visit that may require auscultation or even surgery,
reduce aggressiveness in an animal,
improve an animal's behavior, particularly an aggressive animal or a fearful animal,
produce a relaxing effect on the animal,
help an animal adapt to a new situation, for example upon adoption, when a new animal or baby arrives in its environment, when moving house,
reduce stress-related urinary marking in an animal,
reduce an animal's housebreaking issues,
improve the conditions in which an animal becomes familiar with other animals of the same species,
prevent scratching or destruction of the territory,
reduce noisy manifestations such as whining, barking,
achieve less aggressive, more relaxed and more affectionate behavior in an animal, especially with the owners,
facilitate the training and/or education of an animal, wherein the semiochemical composition is comprised in a diffuser gel as previously defined, comprising:
a gel matrix,
a semiochemical composition,
a solubilizer of said semiochemical composition in said gel matrix,
an organic acid having a pKa greater than 3,
optionally, a preservative,
optionally, a colorant,
optionally, a bittering agent.

### FIGURES

FIG. 1: graph showing the diffusion profile of the semiochemical composition formulated in the diffuser gel in example 1.

FIG. 2 and FIG. 3: graphs comparing the evolution of behaviors FIG. 2 and signs

FIG. 3 in dogs exposed to a diffuser according to the invention or to the Adaptil® Calm diffuser (see example 4). The dotted curve represents the results obtained with the Adaptil® Calm diffuser and the solid curve the results obtained with the diffuser according to the invention.

### EXAMPLES

### Example 1: Preparation of a Diffuser Gel According to the Invention

The pheromone fraction and the isopropanol are stirred in a reactor. In a separate reactor, citric acid, denatonium benzoate, Food yellow 3 colorant, and water are added and heated under stirring to 70° C. When all of the components are dissolved, the carrageenan is added and the mixture is stirred until a homogenous solution at 70° C. is obtained (typically at least 1 hour). The mixture is cooled to a temperature of 50° C. (so as not to damage the esters in the pheromone fraction). The pheromone fraction dissolved in the isopropanol is added to the cooled mixture, followed by benzoisothiazolinone. The final mixture is stirred at 50° C. for about 1 hour and then filled in diffusers.

The diffusers are left at room temperature until a gel has formed. They are then sealed.

| Chemical name | Use | m/m % |
|---|---|---|
| Methyl laurate | Pheromone fraction (total 6%) | 0.118% |
| Methyl myristate | | 0.287% |
| Methyl palmitate | | 1.276% |
| Methyl stearate | | 0.927% |
| Methyl oleate | | 2.113% |
| Methyl linoleate | | 1.280% |
| Propan-2-ol | Active solubilizer & Evaporation | 4.000% |
| | aid | |
| Benzoisothiazolinone | Preservative | 0.180% |
| Citric acid | pH adjuster | 0.050% |
| Carrageenans | Gelling agent | 1.500% |
| Denatonium benzoate | Bittering agent | 0.080% |
| Food Yellow 3 | Colorant | 0.0002% |
| water | | 88.1888% |

### Example 2—Evaluation of the Diffusion Profile of the Semiochemical Composition from the Diffuser Gel in Example 1: Study Conducted Under Controlled Temperature, Humidity and Ventilation Conditions (on Average 22.5° C./54% RH, Room Ventilated Over the Period)

A sufficient number of diffusers to take samples at seven-day intervals for 63 days is placed at TO in a ventilated room under controlled temperature, humidity and ventilation conditions (on average 22.5° C./54% RH, room ventilated over the period). A device is sampled at D 7, 17, 21, 28, 35, 42, 52, 56 and 63 and its contents analyzed by gas chromatography (GC). The samples are prepared as follows:
1. the gel is divided into pieces of about 3 g each,
2. 60 mL of NaOH 30% is added to the 3 g sample until complete dissolution,
3. when all of the gel has dissolved in the NaOH, 30 mL of ethyl acetate is added, the mixture is stirred and left to settle,
4. a sample of the organic phase is taken and analyzed by gas chromatography (detection by FID).

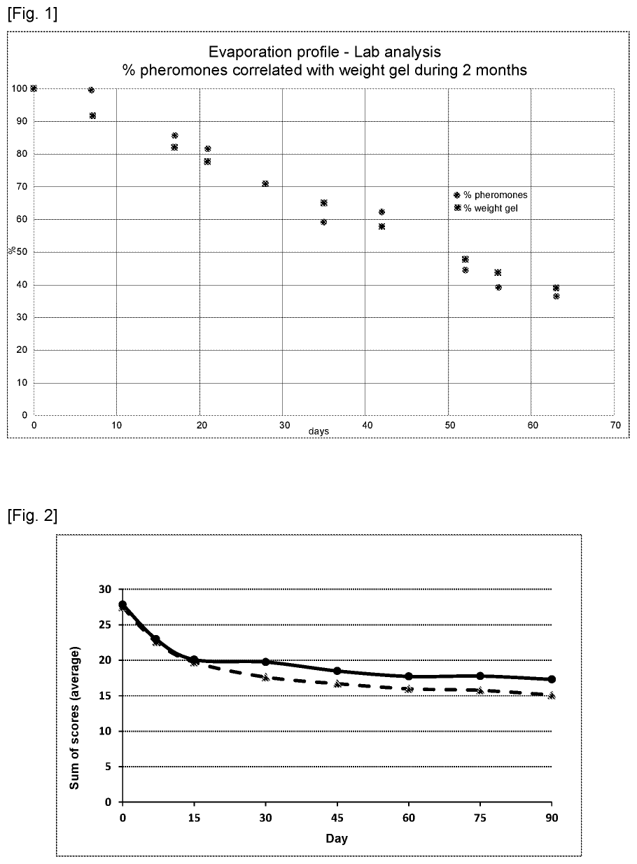

The diffusion profile of the semiochemical composition formulated in the diffuser gel in example 1 is shown on the graph in [FIG. 1].

These results show that the semiochemical composition according to the invention is continuously and regularly released into the environment for more than 60 days when the diffuser gel is in contact with air.

### Example 3—Stability Study of the Semiochemical Composition in the Diffuser Gel in Example 1

Batches of the diffuser gel in example 1 packaged in a closed diffuser were stored under different storage conditions:
3 weeks at 54° C. (CIPAC MT46.3 “Accelerated Storage Procedure” conditions);
6 months at 40° C.

The assay of the pheromones was then performed by chromatography.

The results show that the pheromone content remains constant for the entire storage period and that the diffuser gel according to the invention indeed preserves the semiochemical composition and ensures its storage when the device is air-tight.

### Example 4—Efficacy Perception Study

This study was conducted at the homes of dog owners in France (93 owners) and in Great Britain (103 owners). The households selected for this study have only one dog over four months old living mainly indoors/apartment (outside for less than four hours a day) and with minor behavioral disorders (such as separation anxiety, etc.); they do not have cats. The dogs were not treated for behavioral disorders before and during the study.

Two products were evaluated for three months by groups of independent subjects:
the diffuser containing the diffuser gel in example 1 (a single device used for the three months); and
the commercial Adaptil® Calm product (one starter kit comprising the electric diffuser and one refill+two refills, each refill being used for one month).

The evolution of 17 behaviors:

Whimpers/whines often
Barks excessively or whenever it hears a noise
Growls at people and/or other animals
Destroys/tears up objects, furniture, shoes, toys, trash cans, etc.
Often goes potty in unusual/inappropriate places
Eats its excrements
Pees whenever it has an emotion (happy, fearful, etc.)
Trembles a lot
Often hides or tries to hide
Holds head and/or neck low, tail between the legs
Flinches at the slightest noise
Is afraid in the car
Is afraid of thunderstorms/fireworks
Is afraid of other animals
Does not like to be alone (whimpers, barks, does not eat when we are not
home, etc.)
Remains prostrated (immobility, little activity or play)
Licks its paws for no particular reason (no wounds, injuries, parasites,
etc.)

and 11 signs:
Yawning,
Trembling,
Howling/barking,
Paw licking,
Agitation,
Tail tucked in,
Ears back, hunched/crouching body position,
Hides/tries to hide,

was evaluated by the owners of the dogs in whose environment either of the diffusers had been installed.

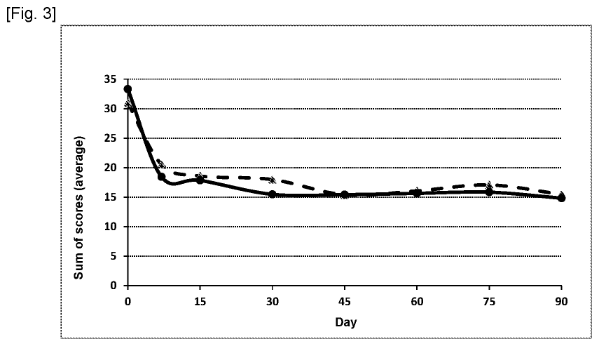

FIGS. 2 and 3 present the results obtained for all of the animals. These results show that the diffuser according to the invention has a three-month efficacy equivalent to that of an electric diffuser in which the refill was replaced every month.

1. A passive diffuser gel of semiochemical compositions for dogs and/or for cats comprising:
a polysaccharide gel matrix,
an appeasing semiochemical composition for dogs and/or for cats, comprising a mixture of volatile fatty acid derivatives,
a solubilizer of said semiochemical composition in said gel matrix, and
an organic acid having a pKa greater than 3.

2. The diffuser gel according to claim 1, wherein the gel matrix is an aqueous carrageenan gel.

3. The diffuser gel according to claim 1, wherein the solubilizer is a C1 to C4 alcohol.

4. The diffuser gel according to claim 1, wherein the organic acid having a pKa of greater than 3 is citric acid.

5. The diffuser gel according to claim 1, wherein the semiochemical composition is a mixture of fatty acid esters comprising:
methyl oleate in a range of from 32% to 38%,
methyl laurate in a range of from 1% to 3%,
methyl stearate in a range of from 13% to 16%,
methyl linoleate in a range of from 18% to 24%,
methyl myristate in a range of from 3% to 7%, and
methyl palmitate in a range of from 18% to 24%.

6. The diffuser gel according to claim 1, wherein the amount of the semiochemical composition is in a range of from 1% to 10% by mass of a total mass of the diffuser gel.

7. The diffuser gel according to claim 1, wherein the diffuser gel has the following composition:
a gel matrix comprising an aqueous carrageenan gel;
a semiochemical composition in range of from 1% to 10%, the semiochemical composition being a mixture of fatty acid esters comprising methyl oleate in a range of from 32% to 38%, methyl laurate in a range of from 1% to 3%, methyl stearate in a range of from 13% to 16%, methyl linoleate in a range of from 18% to 24%, methyl myristate in a range of from 3% to 7%, and methyl palmitate in a range of from 18% to 24%;
a solubilizer of the semiochemical composition in the gel matrix, the solubilizer being in a range of from 2% to 10% selected from C1 to C4 alcohols;
citric acid in a range of from 0.1% to 1%;
a preservative from the isothiazolinone family in a range of from 0.1% to 0.5%;
a colorant; and
denatonium benzoate as a bittering agent.

8. A portable diffuser for controlled release into the environment of a cat or of a dog of a semiochemical composition from a diffuser gel according to claim 2, wherein the portable diffuser comprises a container comprising a diffuser gel-to-air exchange area.

9. The diffuser according to claim 8, wherein the diffuser gel-to-air exchange area comprises one or a plurality of openings through which the semiochemical composition can diffuse.

10. The diffuser according to claim 8, wherein the diffuser gel-to-air exchange area is covered by a removable, airtight lid.

11. The diffuser according to claim 9, wherein an air contact surface at the diffuser gel-to-air exchange area is controlled by adjustable means for closing the openings.

12. A method of providing behavior assistance to an animal using the diffuser gel according to claim 1, wherein the behavior assistance is at least one selected from the group consisting of:
ensuring welfare of the animal, in its environment,
helping the animal handle stressful and/or anxiety-generating situations,
reducing separation-related stress in the animal,
reducing weaning-related stress in the animal,
reducing solitude-related stress in the animal,
reducing stress in the animal before and/or during transport,
reducing stress associated with noise such as thunderstorms or fireworks,
reducing stress associated with a medical condition, a medical treatment, a veterinary visit that may require auscultation or even surgery,
reducing aggressiveness in the animal,
improving the animal's behavior,
producing a relaxing effect on the animal,
helping the animal adapt to a new situation,
reducing stress-related urinary marking in the animal,
reducing the animal's housebreaking issues,
improving conditions in which the animal becomes familiar with other animals of a same species as the animal,
preventing scratching or destruction of the animal's territory,
reducing noisy manifestations, and
achieving less aggressive, more relaxed and more affectionate behavior in the animal.

13. The method according to claim 12, wherein the animal is a cat or a dog, and wherein the behavior assistance is treating stress, anxiety and/or aggressive behavior.

14. The method according to claim 12, wherein the animal is a cat or a dog.

15. The method according to claim 14, comprising:
providing a portable diffuser for controlled release into the environment of the cat or the dog, wherein the portable diffuser comprises a container comprising a diffuser gel-to-air exchange area, and
opening the diffuser gel-to-air exchange area of the portable diffuser.

16. The diffuser gel according to claim 1, further comprising at least one selected from the group consisting of:
a preservative,
a colorant, and
a bittering agent.

17. The diffuser gel according to claim 16, comprising a preservative.

18. The diffuser gel according to claim 16, comprising a colorant.

19. The diffuser gel according to claim 16, comprising a bittering agent.

20. the diffuser gel according to claim 6, comprising about 6% of methyl palmitate.
